(19) United States
(12) Patent Application Publication
(10) Pub. No.: US 2016/0022761 A1
(43) Pub. Date: Jan. 28, 2016

(54) DEREPRESSION OF IMMUNITY IN ONCOLOGICAL AND VIROLOGICAL CONDITIONS BY HIGH MOBILITY GROUP BOX 1 DERIVED PEPTIDES

(71) Applicants: Samuel C. Wagner, San Diego, CA (US); Thomas E. Ichim, San Diego, CA (US); Dimitri N. Theofilopoulos, San Diego, CA (US); Andy J. Kim, San Diego, CA (US)

(72) Inventors: Samuel C. Wagner, San Diego, CA (US); Thomas E. Ichim, San Diego, CA (US); Dimitri N. Theofilopoulos, San Diego, CA (US); Andy J. Kim, San Diego, CA (US)

(21) Appl. No.: 14/807,766

(22) Filed: Jul. 23, 2015

Related U.S. Application Data
(60) Provisional application No. 62/027,903, filed on Jul. 23, 2014.

(51) Int. Cl. A61K 38/08

(52) U.S. Cl. CPC A61K 38/08

(57) ABSTRACT

Disclosed are compositions of peptides derived from the High Mobility Group Box 1 (HMGB1) protein that are useful in suppressing inhibitory activity of T regulatory cells in conditions of cancer and virus infections. Specifically, this application provides means of derepressing immunity in a state of immune suppression through significantly decreasing the number and/or activity of T regulatory cells.

### CROSS-REFERENCE TO RELATED APPLICATION

This application claims the benefit of U.S. Provisional Application No. 62/027,903 filed on Jul. 23, 2014, the contents of which are incorporated by reference herein in its entirety.

### BACKGROUND

The application pertains to the field of immune modulation, more specifically, the application pertains to the field of T regulatory cells. More specifically, the application pertains to modulation of T regulatory cell activity by administration of peptides derived from HMGB1.

The adaptive immune response is commonly initiated via pathogen-associated molecular patterns (PAMPs) that are recognized by Toll-like receptors (TLRs). Dendritic cells (DC) are central for the initiation of adaptive immune responses and are activated by exogenous PAMPs such as lipopolysaccharides (LPS), CpG, or poly(I:C) as well as endogenous signals of tissue and cell damage, sometimes referred to as alarmins or danger signals. Alarmins can take the form of inflammatory cytokines secreted by cells proximal to the site of injury or internal components of damaged cells. Evidence for the latter includes reports that necrotic cell lysates, more specifically heat shock proteins (HSPs) and high mobility group box protein 1 (HMGB1) in the lysates, can induce DC maturation, all of these have demonstrated potent antitumor activities, as well as ability to stimulate vaccine induced tumor inhibition.High Mobility Box Group 1 (HMGB1) was originally described as a nuclear protein that facilitates DNA bending and stabilizes nucleosome formation. HMGB1 contains three domains, including two homologous DNA binding motifs termed A and B boxes, each approximately 80 amino acid long, and a negatively charged C-terminus. In addition to the nuclear functions, HMGB1 is secreted by both macrophages and monocytes after exposure to LPS, TNF-α or IL-1β and through a feedback loop acts back on monocytes by stimulating the synthesis of additional pro-inflammatory cytokines. More recently, HMGB1 was identified as an endogenous alarmin, or damage-associated molecular pattern (DAMP). HMGB1 is released from necrotic cells to trigger inflammation and act as an endogenous adjuvant. Several receptors are implicated in HMGB1 mediated activation of cells, including the receptor for advanced glycation end-products (RAGE), toll like receptor 2 (TLR2), TLR4, TLR9, Mac-1, syndecan-1, receptor-type tyrosine phosphatase-ζ/β), and CD24/Siglec-10.

Structure-function studies have revealed that the proinflammatory domain in HMGB1 maps to the B box domain, which recapitulates the cytokine activity of full-length HMGB1. The current application is based on the previously unknown finding that HMGB1 derived peptides are able to inhibit T regulatory cell generation and activity.

During the last years, CD4+CD25+ regulatory T (Treg) cells have been the subject of intense study. This interest is because their function appears to be critical in the maintenance of peripheral tolerance and regulation of immune responses to non-self Ags. Treg cells can inhibit activation of other T cells and are needed for protection against autoimmune diseases and prevention of rejection of allogeneic transplants. However, immunoregulatory function of Treg cells may hinder the induction of immune responses against cancer and infectious agents. Thus, the presence of Treg cells within tumors may prevent activation of antitumor immune responses favoring tumor growth. This effect suggests that counteracting Treg activity could evoke effective antitumor immunity. Treg cells capable of suppressing the in vitro function of tumor-reactive T cells have been found in humans in tumors such as melanoma, lung, ovary, pancreas and breast cancers as well as hepatocellular carcinoma. Moreover, recent findings suggest that Treg cells infiltrating neoplastic tissues might be associated with a higher death hazard and reduced survival. In infectious diseases, the control exerted by Treg cells may limit the magnitude of effector T cell responses and may result in failure to control infection. Indeed, it has been shown that some viruses, such as hepatitis B, hepatitis C, and HIV, may exploit Treg cells to dampen the antiviral response to favor the persistence of the infection. It is the objective of the current application to provide means of suppressing T regulatory cell activity by administration of HMGB1 derived peptides.

### DESCRIPTION

This application provides means of inhibiting suppressive activity of T regulatory cells. The appropriate dosage of peptides (or derivatives thereof) (or compositions/vaccines/adjuvants including them) for use in accordance with the methods of the this application may depend on a variety of factors. Such factors may include, but are in no way limited to, a subject's physical characteristics (e.g., age, weight, sex), whether the compound is being used as single agent or adjuvant therapy, the type of MHC restriction of the patient, the progression (Le., pathological state) of the infection, and other factors that may be recognized by one skilled in the art.

One skilled in the art would be able, by routine experimentation, to determine an effective, non-toxic amount of peptides (or derivatives thereof) (or compositions/vaccines/adjuvants including them) which would be required to treat the subject. Generally, an effective dosage may be in the range of about 0.0001 mg to about 1000 mg per kg body weight per 24 hours.

Typically, in therapeutic applications, the treatment would be for the duration of the disease state or condition. Further, it will be apparent to one of ordinary skill in the art that the optimal quantity and spacing of individual dosages will be determined by the nature and extent of the disease state or condition being treated, the form, route and site of administration, and the nature of the particular individual being treated. Also, such optimum conditions can be determined by conventional techniques. It will also be apparent to one of ordinary skill in the art that the optimal course of treatment can be ascertained using conventional course of treatment determination tests.

### Example: Treatment with SAFFLFCSE Inhibits T Regulatory Cell Activity

Isolation of human CD4+, CD4+CD25+, and CD4+CD25− T cells was performed from PBMC by using human Treg isolation kits (Miltenyi Biotec), respectively, according to the manufacturer's instructions. The purity of the resulting T cell populations was confirmed to be >95% by flow cytometry.

Inhibitory activity of human Treg cells was measured in in vitro assays of T cell stimulation. CD25-depleted spleen cells (105 cells/well) from human PBMC (105) were stimulated in vitro with the following: anti-human CD3 Ab (BD Pharmingen) or human PBMC (105) from a different donor (to induce a MLR). Cultures were maintained for 72 hours in the presence of SAFFLFCSE peptide at concentrations of 0.01 ug/ml, 0.1 ug/ml and 1 ug/ml. Tritiated thymidine was added at a concentration of 1 microcurie per well for the last 12 hours of culture. Scintillation counting was used to assess proliferation.

>80% inhibition of T regulatory cell suppressive activity was observed in all treatment groups as compared to controls that received scrambled peptide (FFSAFLSEC).

1. A method of inhibiting T regulatory cell activity comprising administering a peptide consisting of the amino acid sequence SAFFLFCSE.

2. The method of claim 1, wherein said T regulatory cell activity is quantified by ability of said T regulatory cell to inhibit proliferation of a CD3 T cell, subsequent to stimulation of said CD3 T cell.

3. The method of claim 1, wherein said T regulatory cell activity is quantified by ability of said T regulatory cell to inhibit Type 1 cytokine production from a CD3 T cell, subsequent to stimulation of said CD3 T cell.

4. The method of claim 2, wherein said stimulation of said CD3 T cell is performed by incubation with a mitogen.

5. The method of claim 3, wherein said stimulation of said CD3 T cell is performed by incubation with a mitogen.

6. The method of claim 1, wherein said peptide comprising the amino acid sequence DPNAPKRPPSAFFLX1X2X3X4 or a derivative thereof, wherein when X1 is alanine (A), glycine (G), or valine (V) then X2 is C, X3 is S and X4 is E; wherein when X2 is alanine (A), glycine (G), or valine (V) then X1 is F, X3 is S and X4 is E; wherein when X3 is alanine (A), glycine (G), or valine (V) then X1 is F, X2 is C and X4 is E; or Wherein when X4 is alanine (A), glycine (G), or valine (V) then X1 is F, X2 is C and X3 is S.

7. The method of claim 6, wherein DPNAPKRPPSAFFLX1X2X3X4 has the amino acid sequence: a. DPNAPKRPPSAFFLX1CSE, b. DPNAPKRPPSAFFLFX1SE, c. DPNAPKRPPSAFFLFCX1E, or wherein X1 is alanine (A), glycine (G), or valine (V).

8. The method of claim 6, wherein DPNAPKRPPSAFFLX1X2X3X4is further mutated so that F at amino acid positions 12 and 13 is changed to S.

9. The method of claim 6, wherein derivative is a fragment of DPNAPKRPPSAFFLX1X2X3X4 having the sequence RPPSAFFLX1X2X3X4, wherein when X1 is alanine (A), glycine (G), or valine (V) then X2 is C, X3 is S and X4 is E; wherein when X2 is alanine (A), glycine (G), or valine (V) then X1 is F, X3 is S and X4 is E; wherein when X3 is alanine (A), glycine (G), or valine (V) then X1 is F, X2 is C and X4 is E; or wherein when X4 is alanine (A), glycine (G), or valine (V) then X1 is F, X2 is C and X3 is S.

10. The method of claim 9, wherein RPPSAFFLX 1X2X3X4 has the amino acid sequence: a. RPPSAFFLX1CSE, b. RPPSAFFLFX1SE, c. RPPSAFFLFCX1E, or d. RPPSAFFLFCSX1, Wherein X1 is alanine (A), glycine (G), or valine (V).

11. The method of claim 10, wherein RPPSAFFLX 1X2X3X4is further mutated so that F at amino acid positions 6 and 7 is changed to S.

12. The method of claim 6, wherein the derivative is a fragment of DPNAPKRPPSAFFLX1X2X3X4 having the amino acid sequence SAFFLX1X2X3X4, wherein when X1 is alanine (A), glycine (G), or valine (V) then X2 is C, X3 is S and X4 is E; wherein when X2 is alanine (A), glycine (G), or valine (V) then X1 is F, X3 is S and X4 is E; wherein when X3 is alanine (A), glycine (G), or valine (V) then X1 is F, X2 is C and X4 is E; or wherein when X4 is alanine (A), glycine (G), or valine (V) then X1 is F, X2 is C and X3 is S.

13. The method of claim 12, wherein SAFFLX1X2X3X4 has the amino acid sequence: a. SAFFLX1CSE, b. SAFFLFX1SE, c. SAFFLFCX1E, or d. SAFFLFCSX1, wherein X1 is alanine (A), glycine (G), or valine (V).

14. The method of claim 12, wherein SAFFLX1X2X3X4 is mutated so that F at amino acid positions 3 and 4 is changed to S.

15. A method of augmenting immune response in a cancer patient comprising the steps of: a) assessing T regulatory cell activity in said cancer patient; b) administering a peptide comprising the amino acid sequence SAFFLFCSE in a pharmaceutically acceptable carrier; and c) assessing T regulatory cell activity after administration to determine need for additional dosing.
